(19) United States
(12) Patent Application Publication
(10) Pub. No.: US 2023/0092433 A1
(43) Pub. Date: Mar. 23, 2023

(54) METAL OXIDE SUNSCREEN FORMULATIONS

(71) Applicant: AMYRIS, INC., Emeryville, CA (US)

(72) Inventors: Christa Lynna MARIANO, Emeryville, CA (US); Ramya VISWANATHAN, Emeryville, CA (US)

(21) Appl. No.: 17/759,770

(22) PCT Filed: Jan. 29, 2021
(86) PCT No.: PCT/US21/15666

§ 371 Date: Jul. 29, 2022

Related U.S. Application Data
(60) Provisional application No. 62/967,817, filed on Jan. 30, 2020.

(51) Int. Cl. A61K 8/27; A61K 8/29; A61K 8/37; A61K 8/31; A61Q 17/04

(52) U.S. Cl. CPC A61K 8/27; A61K 8/29; A61K 8/37; A61K 8/31; A61Q 17/04; A61K 2800/522; A61K 2800/524; A61K 2800/48

(57) ABSTRACT

Provided herein are sunscreen formulations containing a metal oxide sunscreen, squalane, and an antioxidant. The sunscreen formulations provided have increased SPF per unit mass of the metal oxide compared to sunscreen formulations that contain metal oxide without the addition of squalane and antioxidant. Also provided are methods of preventing UV damages to skin involving the application of the sunscreen formulation.

### BACKGROUND

Excessive exposure to sunlight is known to cause a number of health issues including increased incidence of various skin cancers and accelerated aging of the skin. Radiation in the ultraviolet (UV) range (200 nm to 400 nm) has been associated with most of the harmful effects of sun exposure. To mitigate the deleterious effects of sunlight, a number of products have been developed that block or attenuate the energy of UV light. These products block UV radiation and are characterized in their relative effectiveness by a measurement called sun protection factor (SPF). Two distinct types of UV filtering agents have been developed: metal oxide sunscreens and chemical sunscreens. Metal oxides are useful sunscreens however they are characterized by having a dry or chalky application that typically leaves a white residue. In contrast, chemical sunscreens have a better application and are often absorbed with no visible residue. However, recent studies suggest that chemical sunscreens may have worrisome health effects.

UV light has been divided into three distinct classes based on energy content. The most energetic radiation is called UVC (200 nm to 290 nm). Fortunately, UVC is absorbed by the atmosphere and does not pose a significant exposure risk. The second class of UV light, UVB (290 nm to 320 nm), is responsible for most of the acute effects of UV exposure, such as immunosuppression, appearance of erythemas, and induction of skin cancer. The third class of UV light, UVA (320 nm to 400 nm), are the least energetic rays. However, of the three, UVA penetrates the dermis the deepest.

Historically, UVA was not thought to damage skin. Because of that notion, the measurement of SPF focused on the effect of UVB radiation through the measurement of the ability of a product to block the formation of erythema. However, now it is well understood that UVA participates in the photo aging of skin and causes some types of tumors. Accordingly, products with a high SPF but low UVA protection, may put users at risk, because they encourage the exposure to sunlight for a longer time without effectively blocking UVA rays. Today, products having a broader spectrum of protection are favored. These broad-spectrum sunscreen products often contain a mixture of two or more UV filtering compounds. However, the development of broad-spectrum sunscreens have raised additional issues because they often contain mixtures of chemical sunscreens which are thought to pose health risks from use. Accordingly, there is a need for additional broad-spectrum metal oxide based sunscreen formulations that do not have the negative drawbacks of traditional metal oxides sunscreens and which do not have the deleterious health effects of chemical sunscreens.

### BRIEF SUMMARY

In one aspect the disclosure relates to a metal oxide sunscreen formulation which is not chalky, does not leave a white residue, and which is broad-spectrum and high sun protection factor (SPF).

In one embodiment the sunscreen formulation contains a metal oxide, squalane, and one or more antioxidant. In another embodiment the sunscreen formulation has a higher SPF than the metal oxide alone on a per unit mass basis. In yet another embodiment the metal oxide is selected from zinc oxide and titanium oxide. In a further embodiment the antioxidant is ethyl ferulate. In additional embodiments the sunscreen formulation contains a solvent. In a preferred embodiment the solvent is water. In another embodiment the sunscreen formulation contains a chelating agent. In a preferred embodiment the chelating agent is selected from sodium gluconate, sodium phytate, EDTA, tetrasodium glutamate diacetate, trisodium ethylene diamine disuccinate. In yet another embodiment the sunscreen formulation contains a surfactant. In preferred embodiments the surfactant is selected from caprylyl/capryl glucoside, coco-glucoside, isostearic acid, cetearyl glucoside, and arachidyl glucoside. In yet another embodiment the sunscreen formulation contains a humectant. In a preferred embodiment the humectant is selected from glycerin, propanediol, propylene glycol, hexylene glycol, butylene glycol, sorbitol, and xylitol. In a further embodiment the sunscreen formulation contains an emulsion stabilizer. In preferred embodiments the emulsion stabilizer is selected from acacia senegal gum, xanthan gum, cellulose gum, microcrystalline cellulose. In another embodiment the sunscreen formulation contains a viscosity increasing agent. In preferred embodiments the viscosity increasing agent is selected from cetyl palmitate, cetearyl alcohol, methyl dihydroabietate, behenyl alcohol, brassica alcohol, arachidyl alcohol, coconut alcohol, sorbitan palmitate. In yet another embodiment the sunscreen formulation contains an emulsifying agent. In preferred embodiments the emulsifying agent is selected from sorbitan olivate, polyglyceryl-3 polyricinoleate, lecithin, glyceryl stearate, cetearyl olivate. In another embodiment the sunscreen formulation contains an additional emollient in addition to squalane. In preferred embodiments the additional emollient in addition to squalane is selected from caprylic triglyceride and capric triglyceride. In yet another embodiment the sunscreen formulation contains a dispersing agent. In a preferred embodiment the dispersing agent is selected from polyhydroxystearic acid. In an embodiment the sunscreen formulation contains a skin conditioning agent. In preferred embodiments the skin conditioning agent is selected from sodium palmitoyl proline, and nymphaea alba flower extract. In another embodiment the sunscreen formulation contains a preservative. In a preferred embodiment the preservative is selected from phenoxyethanol, benzyl alcohol, hydroxyacetophenone, chlorophensin, potassium sorbate. In another embodiment the sunscreen formulation contains a conditioning agent. In a preferred embodiment the conditioning agent is ethylhexylglycerin.

In certain embodiments the metal oxide is from about 5% w/w to about 25% w/w of the formulation. In other embodiments the squalane is from about 1% w/w to about 25% w/w of the formulation. In yet other embodiments the antioxidant is from about 0.1% w/w to about 2% w/w of the formulation. In embodiments of the invention the metal oxide is about 14% w/w, the squalane is about 5% w/w, and the antioxidant is about 0.7% w/w of the formulation.

In another aspect the invention provides a method of preventing UV damage to skin of a subject comprising applying an effective amount of the sunscreen formulation disclosed herein to the skin of the subject. In an embodiment of the method the sunscreen formulation is applied to the skin prior to exposure to UV light.

### BRIEF DESCRIPTION OF THE DRAWINGS

FIG. 1 is a graph showing the mean absorbance spectra obtained for the blank (control) plates.

FIG. 2 is a graph showing the mean absorbance spectra in UV for the plates with the sunscreen study sample applied, before ultraviolet exposure.

FIG. 3 is a graph showing the mean absorbance spectra in UV for the plates with the applied, after ultraviolet exposure.

FIG. 4 is a graph showing the mean absorbance spectra for blank (control) plates in the analysis of broad-spectrum protection.

FIG. 5 is a graph showing the protection of the sunscreen study sample in the analysis of broad-spectrum protection and calculation of critical wavelength.

### DETAILED DESCRIPTION

The sunscreen formulations disclosed herein rely on metal oxide also known as mineral oxide UV agents as the active agent within the formulation. Suitable metal oxides include zinc oxide and titanium oxide. Zinc oxide is the preferred metal oxide of the formulation.

The metal oxide-based sunscreen formulations disclosed include one or more antioxidants and squalane in addition to the metal oxide sunscreen. The antioxidant in combination with squalane was found to increase the SPF of the formulation compared to a formulation that lacks an antioxidant and squalane. Accordingly, the antioxidant and squalane components were found to potentiate the UV blocking ability of the metal oxide. Antioxidants are compounds that inhibit oxidation. The preferred antioxidant is ethyl ferulate.

As used herein, “squalane” refers to a compound having the following formula:

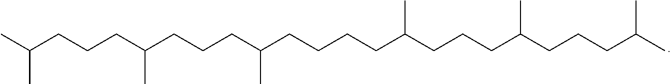 [SMILES: CC(C)CCCC(C)CCCC(C)CCCCC(C)CCCC(C)CCCC(C)C]

The sunscreen formulations of the invention include squalane. Squalane acts as an emollient in the formulation and mitigates the unpleasant properties of metal oxide sunscreen compounds, including: reducing the heavy or tacky feel and reducing or eliminating the chalky finish on application. Surprisingly, the combination of squalane and an antioxidant was also found to increase the SPF of the metal oxide sunscreen relative to the amount of metal oxide in the sunscreen. In certain embodiments squalane comprises from about 1% w/w to about 25% w/w of the formulation. In another embodiment, squalane comprises from about 5% w/w to about 20% w/w of the formulation. In a preferred embodiment the sunscreen formulation comprises about 5% w/w of the formulation.

The sunscreen formulations disclosed herein may also contain one or more solvents. Solvents are used to dissolve various solutes that have one or more functions in the formulation. Solutes dissolved by the solvent may function as chelating agents, surfactants, humectants, emulsion stabilizers, viscosity increasing agents, emulsifying agent, additional emollient in addition to squalane, dispersing agents, skin conditioning agents, preservatives, and conditioning agents. A preferred solvent of the invention is water.

The formulations disclosed herein may contain one or more chelating agents. Chelating agents are chemical compounds that react with metal ions to form stable, water-soluble complexes. Illustrative chelating agents include sodium gluconate, sodium phytate, EDTA, tetrasodium glutamate diacetate, trisodium ethylene diamine disuccinate. A preferred chelating agent of the invention is sodium gluconate.

The formulations disclosed herein may contain one or more surfactants. Surfactants function to lower the surface tension of one or more liquids of the formulation. Illustrative surfactants include caprylyl/capryl glucoside, coco-glucoside, isostearic acid, cetearyl glucoside, and arachidyl glucoside. Preferred surfactants useful in the formulation include caprylyl/capryl glucoside, coco-glucoside, and isostearic acid.

The formulations disclosed herein may also contain one or more humectants.

Humectants are compounds that retain the moisture of the formulation and are typically hygroscopic compounds having multiple hydrophilic groups. Illustrative humectants include glycerin, propanediol, propylene glycol, hexylene glycol, butylene glycol, sorbitol, and xylitol. A preferred humectant of the formulation is glycerin.

The formulations disclosed herein may also contain one or more emulsion stabilizers. Emulsion stabilizers are used to keep the droplets that comprise an emulsion from coalescing. Illustrative emulsion stabilizers include acacia senegal gum, xanthan gum, cellulose gum, microcrystalline cellulose. Preferred emulsion stabilizers of the formulation include acacia senegal gum and xanthan gum.

The formulations disclosed herein may include one or more viscosity increasing agents. Viscosity increasing agents are compounds that act by thickening the formulation and thereby increasing the overall viscosity of the sunscreen formulation. Illustrative viscosity increasing agents include cetyl palmitate, cetearyl alcohol, methyl dihydroabietate, behenyl alcohol, brassica alcohol, arachidyl alcohol, coconut alcohol, sorbitan palmitate. Preferred viscosity increasing agents include cetyl palmitate, cetearyl alcohol, and methyl dihydroabietate.

The formulations disclosed herein may include one or more emulsifying agents or emulsifier. Emulsifying agents are compounds that keep dissimilar chemicals (such as hydrophobic and hydrophilic compounds) from separating in an emulsion. Illustrative emulsifying agents include sorbitan olivate, polyglyceryl-3 polyricinoleate, lecithin, glyceryl stearate, cetearyl olivate. Preferred emulsifying agents include sorbitan olivate, polyglyceryl-3 polyricinoleate, and lecithin.

The formulations disclosed herein may include one or more emollients in addition to squalane. Emollients are substances that soften skin by slowing or preventing the evaporation of water. Squalane functions as an emollient. However, one or more additional emollients may be added to formulation. Suitable emollients in addition to squalane include caprylic triglyceride and capric triglyceride.

The formulations disclosed herein may contain one or more dispersing agents. Dispersing agents are compounds that improve the separation of particles in suspension or in a colloidal dispersion and reduce the settling or agglomeration of particular compounds within the formulation. A preferred dispersing agent of the formulation is polyhydroxystearic acid.

The formulations disclosed herein may contain one or more skin conditioning agents. Suitable skin conditioning agents include sodium palmitoyl proline, nymphaea alba flower extract, and ethylhexylglycerin.

The formulations disclosed herein may contain one or more preservatives. Illustrative preservatives include phenoxyethanol, benzyl alcohol, hydroxyacetophenone, chlorophensin, potassium sorbate. A preferred preservative is phenoxyethanol.

As used herein an “effective amount” means an amount necessary to at least partly attain the desired response, or to delay the onset or inhibit progression or halt altogether, the onset or progression of a particular symptom being treated. The amount varies depending upon the health and physical condition of the subject to be treated, the taxonomic group of subject to be treated, the degree of protection desired, the formulation of the composition, the assessment of the medical situation, and other relevant factors. It is expected that the amount will fall in a relatively broad range that can be determined through routine trials.

As used herein, “subject” or “patient” is an organism that is treated using one of the methods of the present disclosure. In an embodiment, the subject is a mammalian subject, such as a human or a domestic animal.

As used herein, the term “about” will be understood by persons of ordinary skill in the art and will vary to some extent on the context in which is used. If there are uses of the term which are not clear to persons of ordinary skill in the art given the context in which is used, “about” may mean up to plus or minus 20% of the particular term.

As used herein, the term “ointment” may be any commonly known and commercially available ointments.

As used herein “UV” and “ultraviolet radiation” refer to light having a wavelength of from 200 nm to 400 nm.

As used herein “UVA” and “ultraviolet radiation A” refer to light having a wavelength of from 320 nm to 400 nm.

As used herein “UVB” and “ultraviolet radiation B” refer to light having a wavelength of from 290 nm to 320 nm.

As used herein “UVC” and “ultraviolet radiation C” refer to light having a wavelength of from 200 nm to 290 nm.

### EXAMPLES

### Example 1: In Vitro UVA Protection Factor and Critical Wavelength Determination

Study Design and Methods

A sunscreen study sample comprising a metal oxide sunscreen, squalane, and an antioxidant (see Table 1) was prepared. The sun protection factor (SPF) of the sunscreen was measured using an in vitro assay. This in vitro assay is based on PMMA (poly methyl methacrylate) plates having a six micrometer roughened surface (Helioplates HD6 manufactured by Helioscreen).

TABLE 1
Composition of Sunscreen Formulation

| Component | % w/w | Function |
|---|---|---|
| Zinc Oxide | 14% | Sunscreen Agent |
| Squalane | 5% | Emollient |
| Ethyl Ferulate | 0.7% | Antioxidant |
| Water | 48.3% | Solvent |
| Sodium Gluconate | 0.05% | Chelating Agent |
| Caprylyl/Capryl Glucoside | 0.3% | Surfactant |
| Glycerin | 5% | Humectant |
| Acacia Senegal Gum | 0.0825% | Emulsion Stabilizer |
| Xanthan Gum | 0.0675% | Emulsion Stabilizer |
| Cetyl Palmitate | 1% | Viscosity Increasing Agent |
| Cetearyl Alcohol | 2.8% | Viscosity Increasing Agent |
| Coco-Glucoside | 1.2% | Surfactant |
| Sorbitan Olivate | 1.5% | Emulsifying Agent |
| Caprylic/Capric Triglyceride | 15% | Emollient |
| Methyl Dihydroabietate | 2% | Viscosity Increasing Agent |
| Poly hydroxy stearic Acid | 0.4% | Dispersing Agent |
| Polyglyceryl-3 Polycinoleate | 0.2% | Emulsifying Agent |
| Isostearic Acid | 0.2% | Surfactant |
| Lecithin | 0.2% | Emulsifying Agent |
| Sodium Palmitoyl Proline | 0.945% | Skin Conditioning Agent |
| Nymphaea Alba Flower | 0.0045% | Skin Conditioning Agent |
| Extract | | |
| Phenoxyethanol | 0.9% | Preservative |
| Ethylhexylglycerin | 0.1% | Conditioning Agent |

The sunscreen study sample was applied at an amount of approximately 1.3 mg per cm² over a 25 cm² roughened surface area of four PMMA plates. The sunscreen study sample was manually spread across the PMMA plate surfaces with a fingertip that had been previously saturated with the sunscreen formulation. The sample was allowed to rest and dry for 30 minutes in a dark drying chamber kept at 29.1 to 29.2° C.

Control plates were treated similarly except instead of sunscreen study sample a glycerin control was applied. The absorbance spectrum of the control plates was determined in the 290 nm to 400 nm range, measured in 1 nm intervals. Five spectra were obtained from five different points on each plate. The reading areas on each point is 0.79 cm².

The absorbance spectrum of the sunscreen study sample was determined in the 290 nm to 400 nm range, measured in 1 nm intervals, using the control plate as a reference value. Five spectra of the sunscreen study sample were obtained in five different points of the plate. With the mean absorbance spectrum of each plate, the initial in vitro SPF (SPF_{in vitro}) was calculated using the following formula:

$${SPF}_{in vitro} = \frac{{\int }_{λ = 290}^{λ = 400} {E}_{λ} \times {I}_{λ} \times {d}_{λ}}{{\int }_{λ = 290}^{λ = 400} {E}_{λ} \times {I}_{λ} \times {10}^{- {A}_{0 λ}} \times {d}_{λ}}$$

where:

E_{λ}: Erythema Action Spectrum;

I_{λ}: Simulated Spectral Irradiance of the UV range:

A_{0λ}: Mean monochromatic absorbance of the sunscreen formulation, before UV exposure; and

d_{λ}: Increment of wavelength.

Next, the coefficient of adjustment C was determined, used to equal the value of in vitro SPF to the value obtained in the in vivo test. The value of C was calculated, iteratively, to satisfy the condition:

$${SPF}_{in vitro , aj} = \frac{{\int }_{λ = 290}^{λ = 400} {E}_{λ} \times {I}_{λ} \times {d}_{λ}}{{\int }_{λ = 290}^{λ = 400} {E}_{λ} \times {I}_{λ} \times {10}^{- {A}_{0 λ} \times C} \times {d}_{λ}} = {SPF}_{in vivo}$$

where:

E_{λ}: Erythema Action Spectrum;

I_{λ}: Simulated Spectral Irradiance of the UV range:

A_{0λ}: Mean monochromatic absorbance of the sunscreen formulation, before UV exposure;

C: Coefficient of adjustment previously determined in equation; and

d_{λ}: Increment of wavelength.

The value of the coefficient C must be within the range of 0.8 to 1.6, as determined by the control standard. If a value outside the determined range had been obtained, new plates would have been prepared to validate the obtained results.

With these data, the initial UVA-PF (UVA-PF₀) of the product was calculated through the formula:

$$UVA - {PF}_{0} = \frac{{\int }_{λ = 320}^{λ = 400} {P}_{λ} \times {I}_{λ} \times {d}_{λ}}{{\int }_{λ = 320}^{λ = 400} {P}_{λ} \times {I}_{λ} \times {10}^{- {A}_{0 λ} \times C} \times {d}_{λ}}$$

where:

P_{λ}: Persistent Pigmentation Action Spectrum;

I_{λ}: Simulated Spectral Irradiance in the UVA range;

A_{0λ}: Mean monochromatic absorbance of the sunscreen formulation, before UV exposure;

C: Coefficient of adjustment previously determined in equation; and

d_{λ}: Increment of wavelength.

All four PMMA plates containing the sunscreen study sample were exposed to a controlled dose of UV radiation, in order to place the sunscreen study sample under conditions close to the real ones. An irradiator UV Atlas (model Suntest CPS+) equipped with a filter UV Special Glass was used. The sunscreen study sample was exposed to radiation in the UVA, UVB, and visible ranges and the dose was calculated in a way to provide an amount D (J/cm²) of energy in the UVA range calculated as:

D=UVA−PF₀×D₀

In which D₀ is defined by ISO 24443 as a dose of 1.2 J/cm² of UVA radiation.

The temperature of the UV exposure chamber was monitored during the whole process and kept between 28.9° C. and 33.6° C.

After exposing the sunscreen study sample to UV radiation, the absorbance of each one of the four plates was again determined, according to the procedure described above, and five spectra were obtained from each plate. With the mean absorbance spectrum of each plate, the UVA-PF was determined, according to the formula:

$$UVA - PF = \frac{{\int }_{λ = 320}^{λ = 400} {P}_{λ} \times {I}_{λ} \times {d}_{λ}}{{\int }_{λ = 320}^{λ = 400} {P}_{λ} \times {I}_{λ} \times {10}^{- {A}_{λ} \times C} \times {d}_{λ}}$$

where

P_{λ}: Persistent Pigmentation Action Spectrum;

I_{λ}: Simulated Spectral Irradiance in the UVA range;

A_{0λ}: Mean monochromatic absorbance of the sunscreen formulation, after UV exposure;

C: Coefficient of adjustment previously determined in equation; and

d_{λ}: Increment of wavelength.

The ratio SPF/UVA-PF was also calculated, from the value of in vivo SPF.

The calculation of the critical wavelength (λc) was determined for the sunscreen study sample applied to all plates, based on the absorbance spectra after UV exposure. The critical wavelength is another measurement of the sunscreen formulations UVA protection ability, defined as the lower wavelength in which the sunscreen study sample absorbance is equal to 90% of the total absorption, according to the equation:

$$\frac{{\int }_{λ = 290}^{λ = λ c} {A}_{λ}}{{\int }_{λ = 290}^{λ = 400} {A}_{λ}} = 0.9$$

where

λc: Critical wavelength; and

A_{λ}: Mean monochromatic absorbance of the product, after UV exposure.

The reference material S2 was tested according to the same procedure described above. The UVA-PF mean value of the reference material after irradiation must be within the range of between 10.7 and 14.7.

Results

The determination of UVA-PF of the sunscreen study sample was repeated in four plates. The UVA-PF of the sample was calculated through the mean of UVA-PF obtained for each plate. The ratio SPF/UVA-PF and λc of the sunscreen study sample were calculated in the same way. Then, the 95% confidence intervals (CI95%) for the sunscreen study sample and reference material UVA-PF was determined, by using the formulas:

CI95%=x̅±c

being,

$$c = \frac{t \times s}{\sqrt{n}}$$

where:
x̅: Mean UVA-PF;
s: Standard Deviation of the Mean;
CI95%: Lower and upper limits of 95% confidence interval;
n: Number of plates; and
t: value of the Student t distribution, bilateral, for n−1 degrees of freedom and 95% of confidence.

The ratio between c and the UVA-PF corresponds to the CI[%], which is calculated according to the equation below:

$$C I [ \% ] = 100 \times \frac{c}{UVA - PF} .$$

The test can be considered valid if the mean UVA-PF of the reference S2 was in the expected range and the c value was not superior to the 17% of the mean UVA-PF (CI[%]<17.0), for both product and the reference material S2.

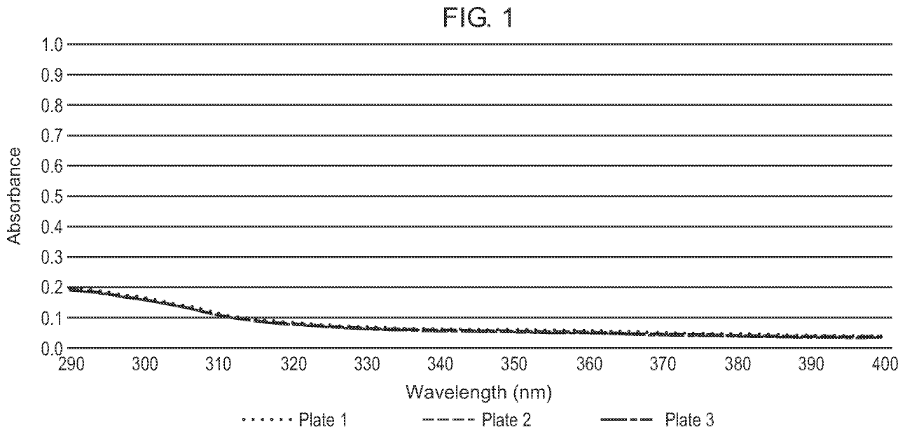

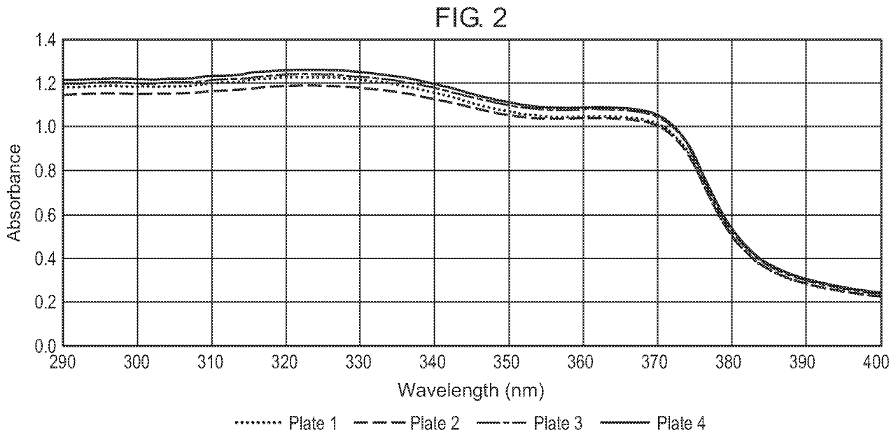

FIG. 1 shows the mean absorbance spectra obtained from the control plates whereas FIG. 2 shows the mean absorbance spectra obtained from all plates with the sunscreen study sample applied. Table 2 shows the respective values of in vitro SPF, the coefficient of adjustment C, and the UVA-PF₀ individually calculated for the plates. The coefficient of adjustment C was calculated to adjust the in vitro SPF to the in vivo value of 30.

TABLE 2
Data of sunscreen study sample application and UVA protection
factors before UVA exposure and irradiated UVA dose.

| | Mass of | | | | |
|---|---|---|---|---|---|
| | Sunscreen | | | | |
| | study | | | In vitro | UVA Dose |
| Plate | sample (mg) | In vitro SPF | C* | UVA-PF₀ | (J/cm²) |
| 1 | 32.6 | 15.2 | 1.3 | 13.7 | 16.4 |
| 2 | 33.0 | 14.1 | 1.3 | 14.2 | 17.0 |
| 3 | 32.7 | 15.8 | 1.2 | 14.2 | 17.0 |
| 4 | 32.7 | 16.4 | 1.2 | 14.1 | 16.9 |
| Mean | 32.8 | 15.4 | 1.2 | 14.0 | 16.8 |
| S.D. | 0.2 | 1.0 | 0.0 | 0.3 | 0.3 |

*C: coefficient of adjustment previously determined in equation.

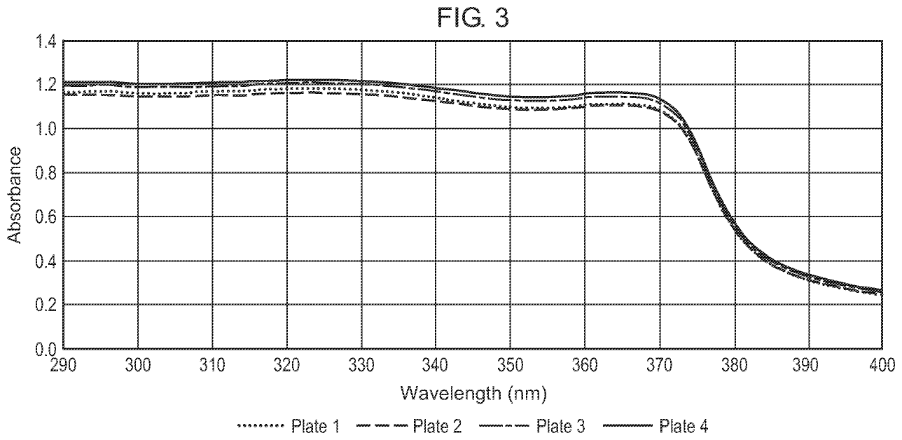

The samples were submitted to UV exposure in an irradiator calibrated every 18 months. The spectral irradiance of the sun simulator in the 320 nm to 400 nm range (UVA) is 78.78 W/m². FIG. 3 shows the mean absorbance spectra of the plates treated with the sunscreen study sample after UV exposure. Table 3 shows the respective values of UVA-PF individually calculated for each plate and Table 4 shows the mean UVA-PF and 95% confidence interval for the sunscreen formulation.

TABLE 3
Final UVA-PF, ratio between SPF/UVA-PF, and critical
wavelength (λc) for the sunscreen formulation.

| Plate | In vitro UVA-PF | SPF/UVA-PF | λc (nm) |
|---|---|---|---|
| 1 | 15.1 | 2.0 | 375 |
| 2 | 16.0 | 1.9 | 375 |
| 3 | 15.7 | 1.9 | 375 |
| 4 | 15.8 | 1.9 | 375 |
| Mean | 15.6 | 1.9 | 375 |
| S.D. | 0.4 | 0.0 | 0.0 |

TABLE 4
Mean UVA-PF and 95% confidence interval.

| | | | CI95% | CI95% | |
|---|---|---|---|---|---|
| Mean UVA-PF(x) | S.D. | C* | x − c | x + c | CI[%] |
| 15.6 | 0.4 | 0.6 | 15.0 | 16.2 | 3.9 |

*c: Factor calculated according to equation presented above.

The mean UVA-PF of the sunscreen study sample was 15.6, with a coefficient of variation of 2.5%. The SPF/UVA-PF mean ratio calculated according to the in vivo SPF was 1.9. The mean critical wavelength (λc) was 375 nm. The c value was inferior to 17% of the mean UVA-PF (CI[%], 17.0%), therefore the test could be considered valid. The reference material S2 presented a mean UVA-PF of 14.0. The obtained UVA-PF values are within the expected range and the c value was inferior to 17% of the mean UVA-PF (IC[%]<17.0%).

### Example 2: In Vivo Evaluation of UVA-PF

Study Design and Methods

Ten female subjects, aged between 20 and 61 years old (mean of 35 years) and all having phototypes III to IV were recruited and enrolled for this study under informed consent. The study inclusion criteria include: being healthy subjects, intact skin on test site, agreement to adhere to the study procedures, ability to give written consent for participating in the study, aged from 18 to 70 years old, phototype II to IV, ITA° between the range of 20° and 41°. Study exclusion criteria include: pregnancy or breastfeeding, skin pathology at application site, type 1 diabetes mellitus, gestational diabetes mellitus, diabetes mellitus with complications, insulin user, presence of dermatosis related to diabetes mellitus, antecedent episodes of hypoglycemia, diabetic ketoacidosis, and/or hyperosmolar coma, immunologic insufficiency, use of systemic corticosteroids or immunosuppressant drugs less than two weeks before the start of the study, current use of antihistamine or anti-inflammation drugs or intent to use these drugs during the study, skin disease, antecedent reaction to the sunscreen study sample components, history of allergies or sensitivity to cosmetics, hygiene products, sunscreens, and/or topical-use products, other diseases or health risks, sun exposure to test site within past four weeks, being part of a study to determine SPF/UVA-PF within the past two months, taking photosensitizing drugs, history of allergic, photoallergic, or toxic reactions to solar exposure, use of self-tanning products within the past month, undergoing artificial tanning, vaccination within 3 weeks of the study, personal or family history of skin cancer, presence of sunburn, scars, and/or active dermal lesions, and any other condition not mentioned that in the investigator's opinion may compromise the study evaluation.

With the subject lying on a stretcher, three sites were demarcated on her back, each site being approximately 30 cm². The sunscreen study sample was applied to one area, while a control was applied to one of the remaining areas, and the third area was left untreated. Approximately 60+/−1.5 mg of sunscreen formulation, corresponding to 2.0 mg/cm² was applied and spread with the aid of a finger cot. Following a rest period of 15 to 30 minutes, the three sites were irradiated. For irradiation, a series of six ultraviolet A radiation (320-400 nm) doses were used, with a 25% variation between each dose.

The pigmentation of the irradiated sites was assessed within a period of from 2 to 24 hours after completing each exposure. On protected and unprotected areas, the observations were done by a trained technician at the same relative moment right after the end of each UVA exposure. The minimum persistent pigmentation dose (MPPD) was defined as a lowest dose of ultraviolet A (320-400 nm) capable of producing the first response with defined borders, appearing in most of the area exposed to UVA radiation, observed 2 to 24 hours after the end of the UVA exposure.

The product UVA-PF for each subject was calculated as the ratio between the MPPD of protected skin (MPPDp) and the MPPD of unprotected skin (MPPDu):

$$UVA - PF = \frac{MPPDp ( protected skin )}{MPPDu ( unprotected skin} .$$

The result was rejected if during the assessment of any of the irradiated areas: 1) there was no change of the pigmentation (underexposure); 2) there was a change on the pigmentation of all areas (overexposure); or 3) if the change progression of the color would not follow a regular sequence.

The UVA-PF determination was repeated in a number of study subjects sufficient for obtaining a minimum of 10 valid results. The mean UVA-PF (x) and the standard deviation (s) for the sunscreen study sample and control were calculated. Next, the 95% confidence intervals for the UVA-PF of the sunscreen study sample and control were determined using the formulas:

$$CI 95 \% = \overline{X} \pm c Where : c = \frac{t \times s}{\sqrt{n}}$$

where:
CI95%: Lower and upper bounds of 95% confidence interval;
n: Number of measurements; and
t: value of the Student t distribution, bilateral, for n−1 degrees of freedom and 95% of confidence.

Results

Ten study subjects completed the study. The data values obtained for each subject are shown in Table 5 and the mean UVA-PF, standard deviation, and 95% confidence interval for the sunscreen study sample and control are shown in Table 6.

TABLE 5
Individual MPPD and UVA-PF results for sunscreen study sample
and control

| | | Study Sample | | Control | |
|---|---|---|---|---|---|
| | MPPDu | MPPDp | Study Sample | MPPDp | Control |
| Subject | (J/cm²) | (J/cm²) | UVA-PF | (J/cm²) | UVA-PF |
| 1 | 386.8 | 221.0 | 12.0 | 239.5 | 13.0 |
| 2 | 483.8 | 221.0 | 9.6 | 299.5 | 13.0 |
| 3 | 483.5 | 257.8 | 11.2 | 299.3 | 13.0 |
| 4 | 483.8 | 322.6 | 14.0 | 299.5 | 13.0 |
| 5 | 483.8 | 165.1 | 7.2 | 239.5 | 10.4 |
| 6 | 604.8 | 257.8 | 9.0 | 299.3 | 10.4 |
| 7 | 483.8 | 322.6 | 14.0 | 239.5 | 10.4 |
| 8 | 483.8 | 322.6 | 14.0 | 299.5 | 13.0 |
| 9 | 483.8 | 206.2 | 9.0 | 299.5 | 13.0 |
| 10 | 483.8 | 322.6 | 14.0 | 299.5 | 13.0 |

TABLE 6
Mean UVA-PF, standard deviation, and 95% confidence
interval for the sunscreen study sample and control.

| | | Stan- | | | | | | |
|---|---|---|---|---|---|---|---|---|
| | Mean | dard | | | | | | |
| | UVA-PF | Devia- | | | | CI % | CI95% | CI95% |
| | (X̅) | tion (s) | n | t | c | (%) | X̅ − c | X̅ + c |
| Sun- | 11.4 | 2.6 | 10 | 2.26 | 1.9 | 16.7 | 9.5 | 13.3 |
| screen | | | | | | | | |
| Study | | | | | | | | |
| Sample | | | | | | | | |
| Control | 12.2 | 1.3 | 10 | 2.26 | 0.9 | 7.4 | 11.3 | 13.1 |

### Example 3: In Vivo Evaluation of SPF

Study Design and Methods

Ten female subjects, aged between 21 and 49 years old (mean of 37 years) and all having phototypes I to III were recruited and enrolled for this study under informed consent. The study inclusion criteria include: being healthy subjects, intact skin on test site, agreement to adhere to the study procedures, ability to give written consent for participating in the study, aged from 18 to 70 years old, phototype I to III, ITA° higher than 28°. Study exclusion criteria include: pregnancy or breastfeeding, skin pathology at application site, type 1 diabetes mellitus, gestational diabetes mellitus, diabetes mellitus with complications, insulin user, presence of dermatosis related to diabetes mellitus, antecedent episodes of hypoglycemia, diabetic ketoacidosis, and/or hyperosmolar coma, immunologic insufficiency, use of systemic corticosteroids or immunosuppressant drugs less than two weeks before the start of the study, current use of antihistamine or anti-inflammation drugs or intent to use these drugs during the study, skin disease, antecedent reaction to the sunscreen study sample components, history of allergies or sensitivity to cosmetics, hygiene products, sunscreens, and/or topical-use products, other diseases or health risks, sun exposure to test site within past four weeks, being part of a study to determine SPF/UVA-PF within the past two months, taking photosensitizing drugs, history of allergic, photoallergic, or toxic reactions to solar exposure, use of self-tanning products within the past month, undergoing artificial tanning, vaccination within 3 weeks of the study, personal or family history of skin cancer, presence of sunburn, scars, and/or active dermal lesions, and any other condition not mentioned that in the investigator's opinion may compromise the study evaluation.

The assessment was initiated with the subject lying on a stretcher, then three areas of 30 cm² each were demarcated on the subject's back. The first area was treated with the sunscreen study sample, the second was treated with a control (a known sunscreen having an SPF 16), and the third was an untreated control. The sunscreen study sample and the sunscreen control were applied with a micro pipette; for both 60 mg of material was applied which corresponds to approximately 2.0 mg/cm². The three sites were then irradiated approximately 15 to 30 minutes after material application. For irradiation, a series of six ultraviolet radiation doses were used, with a 25% variation between each dose (for materials with SPF up to 25) and a 12% variation (for materials with SPF above 25). The series were centered in the expected values of Minimum Erythemal Doses, according to a provisional measurement previously performed. The formula used to calculate the doses was:

D=1.25ⁿ×ₚᵣₒᵥMEDu×SPF #

where:
D: erythema-effective UV dose irradiated;
n: the integer numbers 2, 1, 0, −1, −2, −3 for doses from 1 to 6, respectively;
ₚᵣₒᵥMEDu: provisional minimum erythemal dose for the subject, previously determined; and SPF #: theoretical sun protection factor of the material being tested (equal to 1 for unprotected skin).

For products with an SPF value above 25, the factor used was 1.12 instead of 1.25.

Erthemas were assessed by a trained technician, in a period of 16 to 24 hours after irradiation. The minimal erythemal dose (MED) was defined as the lowest ultraviolet dose capable of generating a non-ambiguous, minimally perceptible erythema.

The product SPFi for each subject was calculated as the ratio between the MED of protected skin (MEDp) and the MED of unprotected skin (MEDu):

$$SPFi = \frac{MEDp ( protected skin )}{MEDu ( unprotected skin )}$$

The result was rejected if, at the assessment of any irradiated area, there was no erythema appearance (sub-exposure), or there was erythema on all subsites (over-exposure), or if the erythema's progression did not follow a regular sequence.

The SPF determination was repeated in a number of study subjects sufficient for obtaining at least 10 valid results. The mean SPF (x) and the standard deviation (s) for the sunscreen study sample and control were calculated. Next, the 95% confidence intervals for the sunscreen study sample and control SPF were determined, by using the formulas:

$$C I 95 \% = X \pm c Where : c = \frac{t \times s}{\sqrt{n}}$$

where:

CI95%: lower and upper bounds of 95% confidence interval;
n: number of measurements; and
t: value of the Student t distribution, bilateral, for n−1 degrees of freedom and 95% of confidence.

Results

The SPF of the sunscreen study sample as determined for each of the ten subjects is shown in Table 7, and the mean SPF of the sunscreen study sample is shown in Table 8.

TABLE 7
Individual static results of MED and SPF for sunscreen study
sample and control.

| | | Study Sample | Study | Control | |
|---|---|---|---|---|---|
| | MEDu | MEDp | Sample | MEDp | Control |
| Subject | (mJ/cm²) | (mJ/cm²) | SPF | (mJ/cm²) | SPF |
| 1 | 29.6 | 1226.0 | 41.4 | 474.3 | 16.0 |
| 2 | 36.1 | 1317.2 | 36.5 | 461.5 | 12.8 |
| 3 | 33.4 | 1085.4 | 32.5 | 534.7 | 16.0 |
| 4 | 20.5 | 747.1 | 36.5 | 327.7 | 16.0 |
| 5 | 39.3 | 1435.1 | 36.5 | 502.8 | 12.8 |
| 6 | 36.7 | 1190.5 | 32.5 | 586.4 | 16.0 |
| 7 | 24.8 | 805.3 | 32.5 | 396.7 | 16.0 |
| 8 | 33.4 | 1370.1 | 41.0 | 427.1 | 12.8 |
| 9 | 41.0 | 1494.1 | 36.5 | 655.4 | 16.0 |
| 10 | 36.1 | 1173.0 | 32.5 | 577.8 | 16.0 |

TABLE 8
Mean static SPF, standard deviation, and 95% confidence
interval for the sunscreen study sample and control.

| | | Stan- | | | | | | |
|---|---|---|---|---|---|---|---|---|
| | Mean | dard | | | | | | |
| | UVA-PF | Devia- | | | | CI % | CI95% | CI95% |
| | (X̅) | tion (s) | n | t | c | (%) | X̅ − c | X̅ + c |
| Sun- | 35.8 | 3.4 | 10 | 2.3 | 2.4 | 6.8 | 33.4 | 38.2 |
| screen | | | | | | | | |
| Study | | | | | | | | |
| Sample | | | | | | | | |
| Control | 15.0 | 1.6 | 10 | 2.3 | 1.1 | 7.4 | 13.9 | 16.1 |

### Example 4: Broad Spectrum UV Analysis

According to the FDA standard—2011, UV transmittance values of blank plate and product plate should be obtained first then converted into absorbance values so they can be inserted into the equations for calculating critical wavelength. The absorbance spectrum of blank PMMA plates with glycerin applied in the 290 nm to 400 nm range was determined at 1 nm intervals. An amount of approximately 0.75 mg/cm² of the sunscreen study sample was applied to three PMMA plates with roughened surface, spreading manually with a fingertip to obtain a film coating that is visually even. The plate with the sunscreen study sample was left to rest for a minimum of fifteen minutes protected from light in the interior of a drying chamber with the temperature controlled to be between 28.8° C. to 29.2° C.

All three PMMA plates containing the sunscreen study sample were exposed to a controlled dose of UV radiation, in order to place the product under conditions close to real ones. The sunscreen study sample was exposed to radiation in the UVA, UVB, and visible ranges providing an amount of erythemal effective energy of 800 J/m²eff, equivalent to 4MED.

After the irradiation of the sunscreen study sample containing plates, the absorbance of each one of the three sample plates was determined in the range of 290 nm to 400 nm, in intervals of 1 nm, using a plate treated with glycerin as a reference.

The critical wavelength (λc) was determined for the sunscreen study sample applied on all plates, based on the absorbance spectra after UV irradiation. The critical wavelength is defined as the lowest wavelength at which the product absorbance is equal to 90% of the total absorption, according to the equation:

$$\frac{{\sum }_{λ = 290}^{λ = λ c} {A}_{λ}}{{\sum }_{λ = 290}^{λ = 400} {A}_{λ}} = 0.9$$

where:
λc: critical wavelength; and
A_{λ}: mean monochromatic absorbance of the sunscreen study sample in the wavelength λ.
The critical wavelength value (λc) of the sunscreen study sample is the mean of the individual values of each of the three plates. In order to label a sunscreen product as providing broad-spectrum protection, the mean critical wavelength must be equal to or greater than 370 nm.

### Results

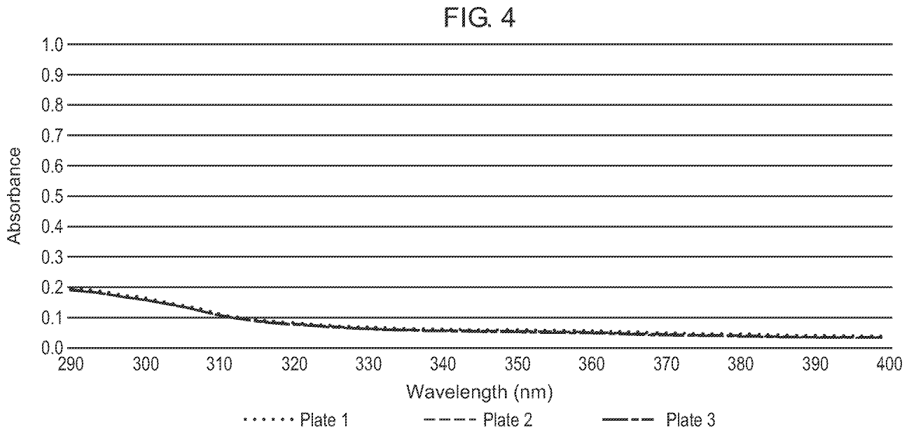

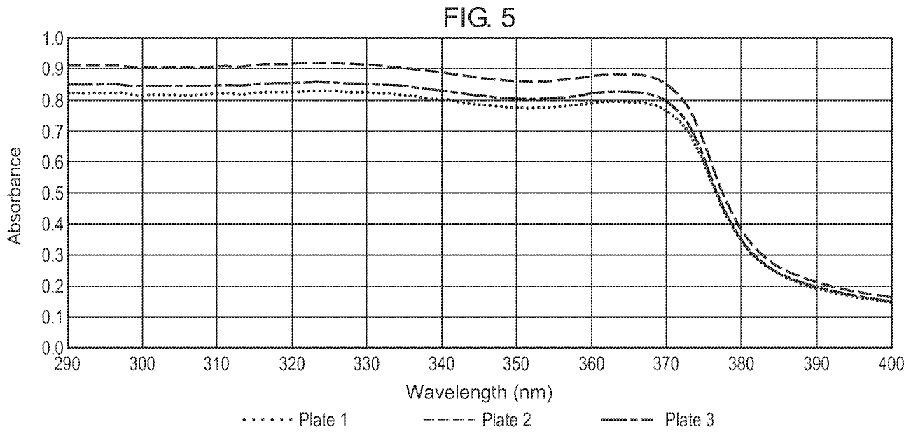

FIG. 4 shows the mean absorbance spectra obtained for all the blank plates whereas FIG. 5 shows the mean absorbance spectra of the sunscreen study sample treated plates after UV irradiation. Table 9 presents the sunscreen study sample application data and the critical wavelength (λc) for each plate with sunscreen study sample applied.

TABLE 9
Data of the sunscreen study sample and critical wavelength for
each plate with sample applied.

| Plate | Mass of sample (mg) | λc (nm) |
|---|---|---|
| 1 | 18.4 | 373 |
| 2 | 19.2 | 373 |
| 3 | 18.1 | 373 |
| Mean | 18.6 | 373 |
| Standard Deviation | 0.6 | 0.0 |

The critical wavelength of the sunscreen study sample was calculated to be 373 nm.
Accordingly, the sunscreen study sample formulation offers a broad-spectrum protection.

### Example 5: SPF Determination Using the FDA Guide on Labeling and Effectiveness Testing: Sunscreen Drug Products for Over-The-Counter Human Use

Ten female subjects, aged between 21 and 52 years old (mean of 38 years) and all having phototypes I to III were recruited and enrolled for this study under informed consent. The study inclusion criteria include: being healthy subjects, intact skin on test site, agreement to adhere to the study procedures, ability to give written consent for participating in the study, aged from 18 to 70 years old, phototype I to III, ITA° higher than 28°. Study exclusion criteria include: pregnancy or breastfeeding, skin pathology at application site, type 1 diabetes mellitus, gestational diabetes mellitus, diabetes mellitus with complications, insulin user, presence of dermatosis related to diabetes mellitus, antecedent episodes of hypoglycemia, diabetic ketoacidosis, and/or hyperosmolar coma, immunologic insufficiency, use of systemic corticosteroids or immunosuppressant drugs less than two weeks before the start of the study, current use of antihistamine or anti-inflammation drugs or intent to use these drugs during the study, skin disease, antecedent reaction to the sunscreen study sample components, history of allergies or sensitivity to cosmetics, hygiene products, sunscreens, and/or topical-use products, other diseases or health risks, sun exposure to test site within past four weeks, being part of a study to determine SPF/UVA-PF within the past two months, taking photosensitizing drugs, history of allergic, photoallergic, or toxic reactions to solar exposure, use of self-tanning products within the past month, undergoing artificial tanning, vaccination within 3 weeks of the study, personal or family history of skin cancer, presence of sunburn, scars, and/or active dermal lesions, and any other condition not mentioned that in the investigator's opinion may compromise the study evaluation.

The assessment was initiated with the subject lying on a stretcher, then, five areas of 30 cm² each were demarcated on the subject's back. First, the sunscreen study sample was applied with the help of a micropipette, in an amount of approximately 60 mg corresponding to 2.0 mg/cm², and uniformly spread by trained technicians, with the aid of a finger cot. After a minimum period of 15 minutes, the site with the sunscreen study sample applied was irradiated. The site that had no product applied was also irradiated. The application of samples, exposure to UV radiation, and definitions of MED were performed under stable conditions, with the room's temperature between 18° C. and 26° C.

A series of six doses of B+A ultraviolet irradiation (290 nm to 400 nm) was carried out, with 25% of variation between each dose, for products with SPF up to and including 8. For product with SPF between SPF 8 and 15, a 20% variation was used, while a 15% variation was used for products with SPF above 15. The series of doses were centered in the third output, which must equal the initial MEDu (previously determined) multiplied by the expected SPF value.

Erythemas were assessed by a trained technician, in a period of 16 to 24 hours after irradiation. The minimum erythemal dose (MED) was defined as the lowest ultraviolet dose capable of generating a non-ambiguous, minimally perceptible erythema.

The static SPFi of the sunscreen study sample for each subject was calculated as the ratio between the MED of protected skin (tpMEDp) and the MED of unprotected skin (MEDu), according to the equation:

$$SPFi = \frac{MEDp ( protected skin )}{MEDu ( unprotected skin )}$$

The SPF determination was repeated in a number of study subjects sufficient for obtaining a minimum of 10 valid results. The mean SPF (X̅) and the standard deviation (s) for sunscreen study sample and control were calculated. Next, the final SPF of the sunscreen study sample and control were determined using the formulas:

$$Final SPF = SPF - ( t \times SE ) Where SE = \frac{s}{\sqrt{n}}$$

where:
S̅P̅F̅: mean of SPFi values;
n: number of measurements; and
t: value of the Student t distribution, bilateral, for n−1 degrees of freedom and 95% of confidence.

The labeled SPF of the sunscreen study sample is defined as the higher integer number lower than the value of the final SPF.

### Results

Table 10 shows the results of the study for each individual subject and Table 11 shows the mean values for the sunscreens study sample and the control.

TABLE 10
Individual static results of MED and static SPF for product
and control.

| | | Study Sample | Study | Control | |
|---|---|---|---|---|---|
| | MEDu | tpMEDp | Sample | ssMEDp | Control |
| Subject | (mJ/cm²) | (mJ/cm²) | SPFi | (mJ/cm²) | SPFi |
| 1 | 36.1 | 1288.3 | 35.7 | 512.2 | 14.2 |
| 2 | 41.8 | 1614.7 | 38.7 | 741.5 | 17.8 |
| 3 | 33.4 | 1032.1 | 30.9 | 534.7 | 16.3 |
| 4 | 20.5 | 839.8 | 41.0 | 333.9 | 16.3 |
| 5 | 36.7 | 1307.6 | 35.7 | 519.8 | 14.2 |
| 6 | 39.3 | 1403.7 | 35.7 | 558.1 | 14.2 |
| 7 | 33.4 | 1192.2 | 35.7 | 474.0 | 14.2 |
| 8 | 24.8 | 884.5 | 35.7 | 532.7 | 21.5 |
| 9 | 41.0 | 1461.4 | 35.7 | 667.7 | 16.3 |
| 10 | 36.1 | 1115.3 | 30.9 | 512.2 | 14.2 |

TABLE 8
Mean static SPF, standard deviation, and 95% confidence
interval for the sunscreen study sample and control.

| | | Stan- | | | | |
|---|---|---|---|---|---|---|
| | Mean | dard | | | | |
| | SPF | Devia- | | | | |
| | (S̅P̅F̅) | tion (s) | n | t | SE | SPF_{final} |
| Sun- | 35.5 | 3.0 | 10 | 2.3 | 1.0 | 33 |
| screen | | | | | | |
| Study | | | | | | |
| Sample | | | | | | |
| Control | 15.9 | 2.4 | 10 | 2.3 | 0.7 | 14 |

1. A sunscreen formulation comprising a metal oxide, squalane, and one or more antioxidant.

2. The sunscreen formulation of claim 1, wherein the sunscreen formulation has a higher sun protection factor (SPF) than the metal oxide alone on a per unit mass basis.

3. The sunscreen formulation of claim 1, wherein the metal oxide is selected from zinc oxide and titanium oxide.

4. The sunscreen formulation of claim 1, wherein the antioxidant is ethyl ferulate.

5. The sunscreen formulation of claim 1, further comprising a solvent.

6. The sunscreen formulation of claim 5, wherein the solvent is water.

7. The sunscreen formulation of claim 1, further comprising a chelating agent.

8. The sunscreen formulation of claim 7, wherein the chelating agent is selected from sodium gluconate, sodium phytate, EDTA, tetrasodium glutamate diacetate, trisodium ethylene diamine disuccinate.

9. The sunscreen formulation of claim 1, further comprising a surfactant.

10. The sunscreen formulation of claim 9, wherein the surfactant is selected from caprylyl/capryl glucoside, coco-glucoside, isostearic acid, cetearyl glucoside, and arachidyl glucoside.

11. The sunscreen formulation of claim 1, further comprising a humectant.

12. The sunscreen formulation of claim 11, wherein the humectant is selected from glycerin, propanediol, propylene glycol, hexylene glycol, butylene glycol, sorbitol, and xylitol.

13. The sunscreen formulation of claim 1, further comprising an emulsion stabilizer.

14. The sunscreen formulation of claim 13, wherein the emulsion stabilizer is selected from acacia senegal gum, xanthan gum, cellulose gum, microcrystalline cellulose.

15. The sunscreen formulation of claim 1, further comprising a viscosity increasing agent.

16. The sunscreen formulation of claim 15, wherein the viscosity increasing agent is selected from cetyl palmitate, cetearyl alcohol, methyl dihydroabietate, behenyl alcohol, brassica alcohol, arachidyl alcohol, coconut alcohol, sorbitan palmitate.

17. The sunscreen formulation of claim 1, further comprising emulsifying agent.

18. The sunscreen formulation of claim 17, wherein the emulsifying agent is selected from sorbitan olivate, polyglyceryl-3 polyricinoleate, lecithin, glyceryl stearate, cetearyl olivate.

19. The sunscreen formulation of claim 1, further comprising an additional emollient in addition to squalane.

20. The sunscreen formulation of claim 19, wherein the additional emollient in addition to squalane is selected from caprylic triglyceride and capric triglyceride.

21. The sunscreen formulation of claim 1, further comprising a dispersing agent.

22. The sunscreen formulation of claim 21, wherein the dispersing agent is selected from polyhydroxystearic acid.

23. The sunscreen formulation of any one of claim 1, further comprising a skin conditioning agent.

24. The sunscreen formulation of claim 23, wherein the skin conditioning agent is selected from sodium palmitoyl proline, and nymphaea alba flower extract.

25. The sunscreen formulation of claim 1, further comprising a preservative.

26. The sunscreen formulation of claim 25, wherein the preservative is selected from phenoxyethanol, benzyl alcohol, hydroxyacetophenone, chlorophensin, potassium sorbate.

27. The sunscreen formulation of claim 1, further comprising a conditioning agent.

28. The sunscreen formulation of claim 27, wherein the conditioning agent is ethylhexylglycerin.

29. The sunscreen formulation of claim 1, wherein the metal oxide is from about 5% w/w to about 25% w/w of the formulation.

30. The sunscreen formulation of claim 1, wherein the squalane is from about 1% w/w to about 25% w/w of the formulation.

31. The sunscreen formulation of claim 1, wherein the antioxidant is from about 0.1% w/w to about 2% w/w of the formulation.

32. The sunscreen formulation of claim 1, wherein the metal oxide is about 14% w/w, the squalane is about 5% w/w, and the antioxidant is about 0.7% w/w of the formulation.

33. A method of preventing UV damage to skin of a subject comprising applying an effective amount of the sunscreen formulation of claim 1 to the skin of the subject.

34. The method of claim 33, wherein the sunscreen formulation is applied to the skin prior to exposure to UV light.
